(19) United States
(12) Patent Application Publication
(10) Pub. No.: US 2012/0077198 A1
(43) Pub. Date: Mar. 29, 2012

(54) Compositions And Methods For Cancer Testing

(75) Inventors: Nicolas Wyhs, Owings Mills, MD (US); Gabe Foster, Waltham, MA (US); Mukundhan Ramaswami, Waltham, MA (US); Christopher Sears, Waltham, MA (US)

(73) Assignee: AMBERGEN, INC

(21) Appl. No.: 13/185,795

(22) Filed: Jul. 19, 2011

Related U.S. Application Data
(60) Provisional application No. 61/369,221, filed on Jul. 30, 2010.

(51) Int. Cl. C12Q 1/68

(52) U.S. Cl. 435/6.12

(57) ABSTRACT

Methods and compositions which provide a gene expression-based prognostic signature of cancer relapse and prediction of metastatic cancer are described, and in particular methods to predict colorectal cancer (CRC) recurrence and chemosensitivity.

### FIELD OF THE INVENTION

The present invention contemplates methods and compositions which provide a gene expression-based prognostic signature of cancer relapse and prediction of metastatic cancer, and in particular colorectal cancer (CRC) recurrence and chemosensitivity.

### BACKGROUND

There have been other attempts at creating a gene expression-based prognostic signature of relapse or response to therapy, but none of these have had much success. Most studies have attempted to correlate an expression signature to a specific histopathological phenotype. Our current understanding of clinical heterogeneity hints at why this may have been unsuccessful. It would be difficult to find a solid set of several genes indicative of certain morphological features, when the same feature could have arisen from several different molecular mechanisms. Other attempts have also had poor experimental designs, yielding bulky signatures of hundreds of genes. See e.g. Kwon et al. Dis Colon Rectum 2004 February; 47(2):141-52. In a clinical setting, it is neither time- nor cost-effective to test every CRC patient for this many biomarkers.

### SUMMARY OF THE INVENTION

We are avoiding these pitfalls through rigorous experimental design and quality assurance measures performed at every step. Our layered approach uses two different population cohorts for the discovery phase of signature development, as well as two tissue types: fresh-frozen and formalin-fixed paraffin-embedded. We refer to literature references to prioritize our genes of interest, and apply innovative changes to our sample preparation protocol. This produces a much more robust signature, and with our focus on translational medicine, a more clinically applicable test.

For formalin-fixed paraffin-embedded (FFPE) samples, we have modified the Sample Extraction procedure described in Ambion's RecoverAll™ Total Nucleic Acid Isolation Kit for FFPE instruction manual. In particular, certain modifications have been made to improve RNA quality and yield in colorectal cancer FFPE tumor tissue sections. In one embodiment, RNA elution is performed using high temperature solutions (>80° C. and up to 95° C.). This high temperature elution, while generating a better overall yield of RNA, creates certain difficulties. The high temperature will cause air inside the pipette tip to expand, and therefore unexpectedly expel aspirated water if the pipette tip is heated too much. This is avoidable by inserting as little of the pipette tip's surface into the solution (e.g. water) as possible, and moving quickly to the Filter Cartridge. Moreover, a great amount of fluid needs to be used for the elution, since the higher temperature will result in some vapor loss. Thus, the present invention contemplates, in one preferred embodiment, two 50 ul (or one 100 ul) high temperature elutions in place of the low temperature elutions taught in the Ambion protocol.

Formaldehyde creates cross-links between proteins, which maintains tissue structure, and cross-links between proteins and nucleic acids, which become trapped and chemically modified. In addition, the embedding process infiltrates tissues with paraffin and requires high temperatures for a prolonged period of time, causing the RNA molecules to undergo further modifications and fragmentation. Older samples, which are often most valuable for prognostic studies, also undergo the greatest nucleic acid degradation. Using a modification to the Ambion protocol, we found it was possible to obtain usable RNA template from FFPE tissue even as old as 5 years. Briefly, this procedure involves incubating for short time periods at an elevated temperature the RNA isolated from FFPE tissue, which disrupts a large proportion of cross-links, releasing sufficient amounts of template to be usable for downstream applications.

While the above-described modifications in the sample preparation phase improve RNA yield and quality, the present invention also contemplates an assay design to ensure control over variability in the actual assay. In one embodiment, the assay is an RT-PCR assay, e.g. in a 384-well format with ABI 7900 HT system. In one embodiment, the assay is a real time PCR assay using the Rox dye. To ensure control over variability, the present invention contemplates, in one embodiment, the use of “spike in” controls comprising oligonucleotides that have no homology to the human genome. Ideally one or more of them are included in every reaction in a known quantity, and then measured with probes from ABI. This lets us observe any potential plate-to-plate reaction efficiency variability. In one embodiment, a spike in control is contemplated comprises a portion of the nucleic acid sequence encoding RNA polymerase II 140 kD subunit from Drosophila Melanogaster, e.g. an oligo of the sequence:

(SEQ ID NO: 1)
ccttccccgatcacaatcagagtccgcgtaacacctatcaaagcgctatgggtaagcaagctatgggcgtttatattaccaacttc
cacgtgcgtatgga.

In another embodiment, a spike in control is contemplated comprises a portion of the nucleic acid sequence encoding Ribosomal protein L32 from Drosophila Melanogaster, e.g. an oligo of the sequence:

(SEQ ID NO: 2)
agcgcaccaagcacttcatccgccaccagtcggatcgatatgctaagctgtcgcacaaatggcgcaagcccaagggtatcgac
aacagagtgcgtcgacg

In another embodiment, a spike in control is contemplated comprises a portion of the nucleic acid sequence encoding the ubiquitin family protein RAD23-3 from Arabidopsis, e.g. an oligo of the sequence:

(SEQ ID NO: 3)
acctgcagcagcacccgcaagtggtcctaatgcaaatccgttagatctcttcccacagggcttgccaaatgttggaggaaatcct
ggtgctggaacacttgacttcttgc.

In one embodiment, all three of these control oligos are employed in the assay.

The present invention also contemplates methods and compositions for reducing RNA degradation. In this regard, amplicon size of the TaqMan gene expression assays is an important consideration. We minimize this effect by designing TaqMan assays that produce the smallest available amplicon size (e.g. preferably between 60-150 bases in length). In addition, since RNA degradation normally begins at the 5′-end of transcripts, we choose probes directed toward the 3′-ends of genes. In order to monitor the level of degradation of the samples, we utilize three GAPDH probes of differing amplicon sizes; the amount of degradation can be inferred by comparing the RT-PCR output of the larger GAPDH amplicons to that of the shorter GAPDH amplicon.

Finally, it is also useful to modify Applied Biosystems RQ Manager software. This is described in co-pending U.S. application Ser. No. 61/331,527, hereby incorporated in its entirety.

The present invention contemplates the above-described methods and compositions which provide a gene expression-based prognostic signature of cancer relapse and prediction of metastatic cancer, and in particular colorectal cancer (CRC) recurrence and chemosensitivity.

In one embodiment, the present invention contemplates a method of improving human RNA yield, comprising: providing human RNA released from formalin-fixed paraffin-embedded colorectal cancer tissue; and recovering said RNA by solid phase extraction on a filter comprising i) loading the RNA on said filter, ii) (optionally) washing the filter, and iii) eluting the RNA, wherein said eluting comprises applying an aqueous solution to said filter, wherein said solution has been heated above 80° C. (and more preferably, it has been heated to approximately 95° C.). In one embodiment, said filter is part of a filter cartridge (e.g. a filter comprising silica). In a preferred embodiment, the yield of RNA eluted from said filter with said heated solution is higher than achieved with an unheated solution. In a preferred embodiment, the volume of solution added to the filter is between 50 and 100 microliters.

The RNA obtained by the above-described method can be used in a variety of assays. In a preferred embodiment, said RNA is utilized in an RT-PCR assay. In a preferred embodiment, said RT-PCR assay generates amplicons between 60 and 150 bases in length. In a preferred embodiment, said RT-PCR assay generates amplicons from non-human control sequences (e.g. non-human control sequences selected from the group consisting of SEQ ID NOS: 1-3.)

In one embodiment, the present invention contemplates a method of controlling for variability, comprising: providing human RNA released from formalin-fixed paraffin-embedded colorectal cancer tissue; and utilizing said RNA in an RT-PCR assay with spike in controls. In one embodiment, said RT-PCR assay generates amplicons from said spike in controls, e.g. non-human control sequences (e.g. non-human control sequences are selected from the group consisting of SEQ ID NOS: 1-3.)

### BRIEF DESCRIPTION OF THE DRAWINGS

FIG. 1 shows a comparison of one embodiment of our three spike in controls (A-RpII140, B-RpL32, C-RAD23-3) with a endogenous control (D-GAPDH) over several samples. When evaluating GAPDH (FIG. 1B), it is clear that a significant portion of the variability that would be attributed to plate variances is, in fact, due to individual variation in expression. This means that there is no effective way, using endogenous genes, to measure inter-plate variability. Therefore, to control for non-sample assay variability, we find it important to include non-sample controls, i.e. spike in controls (FIG. 1A).

### GENERAL DESCRIPTION OF THE INVENTION

An indication of the urgent need for an effective CRC prognostic is reflected in the fact that 70% of untreated CRC patients do not recur (e.g. do not develop non-local metastasized CRC [mCRC]) after a 5-year period. However, the 5-year survival rate for the remaining 30% of CRC patients who do recur is only 10%, much lower than the survival rate among other common cancers which frequently recur, such as prostate (32%) and breast (27%) (see the National Cancer Institute SEER Cancer statistics, 1996-2003 data). Clearly, early identification of the population of CRC patients most likely to recur, combined with the ability to predict response to various treatments, would significantly improve outcomes for many CRC patients.

The most common treatment for post-surgical CRC patients is chemotherapy, including regimens such as 5-fluorouracil (5-FU) in conjunction with other drugs such as leucovorin and oxaliplatin (FOLFOX) or folic acid and irinotecan (FOLFIRI). More recently, monoclonal antibodies such as cetuximab and panitumumab, which are targeted against the extracellular ligand binding site of EGFR, have been introduced. External beam radiation treatment is also often used either alone or in conjunction with chemotherapy. However, such treatment can entail severe side-effects. For example, acute and chronic neuropathy, hypersensitivity reactions, diarrhea, neutropenia, and hand-foot syndrome often occurs in cases of 5-FU-related treatment, while radiation can cause additional cancer and sterility. In contrast, an effective CRC prognostic assay which successfully identifies those patients with CRC most likely to recur, and then correctly predicts their response to various therapies, would limit unnecessary treatment and increase the number of patients with positive outcomes.

The major approach taken in this project is based on measuring the expression level of a panel of genes in post-surgical CRC tumor samples to predict divergent tumor development and response to standard drug therapy and radiation treatment (FIG. 3). This approach is supported by numerous studies which demonstrate that microarray technologies, which exploit genetic characteristics rather than histopathological differences, provide more accurate tumor classification Importantly, microarray data has also made it possible to elucidate some of the molecular mechanisms underlying tumorigenesis in a variety of cancers.

The most common post-surgical protocol currently followed for monitoring patients for recurrence of colorectal cancer is based on the American Joint Committee on Cancer (AJCC) TNM staging (tumor, node, metastasis). After the TNM has been scored, this information is used to determine a stage for the tumor (I-IV) along with various subcategories. In stage II, one the most common stages found after surgery in CRC patients, the tumor has usually penetrated the muscularis muscosa and may have also reached the muscularis propria, but not spread to lymph nodes or distant sites. Unfortunately, the use of TNM at stage II for prediction of recurrence or determining response to a particular therapy is unreliable, yet remains the current clinical standard.

Using the statistical analysis package PRAXIS™, we were able to identify 200 potential genes which correlated with recurrence and 5-FU response (separate gene sets) based on microarray data. By performing RT-PCR analysis of these genes on an independent cohort of FFPE samples, as detailed below, we were able to identify a much smaller set of genes (total of 8) highly correlated with recurrence and response to 5-FU (independent sets). It must be stressed that FFPE samples, while the most common sample available, present challenges. The samples are subject to RNA degradation. Therefore, the genes found important from the fresh frozen microarray work, may not be the best genes for RT-PCT from FFPE samples.

In a preferred embodiment, the present invention contemplates utilizing Ambion's RecoverAll™ Total Nucleic Acid Isolation Kit, which is itself optimized for nucleic acid recovery from formalin-fixed paraffin-embedded tissues, with additional optimizations (as set forth herein) to alleviate some of the chemical modifications induced upon the tissue during fixation and do achieve better yields. The extracted RNA is then quantified, reverse transcribed, and analyzed using the Applied Biosystems (Foster City, Calif.) ABI 7900-HT ‘TagMan’ machine, which is the industry standard in real-time PCR equipment. Our measurements utilize the Taqman Low Density Array (TLDA) platform. This platform based on a 384-well microfluidic card prefilled with probes allows us to minimize both the amount of sample necessary and potential user error while minimizing the need for liquid-handling robots or multichannel pipettors.

In one embodiment, the RNA is recovered from formalin-fixed, paraffin-embedded CRC tumor tissue by removal of the paraffin and tissue digestion with protease(s). This released RNA is recovered by solid phase extraction onto a filter (e.g. silica filter) cartridge (Ambion) by loading successive aliquots (e.g. 700 ul) into the plastic device containing the filter (i.e. the filter cartridge), which is inserted into a (e.g. 2 mL) collection tube. The assembly is centrifuged in a microcentrifuge and the filtrate decanted from the collection tube into a waste container. The filter cartridge is then washed (e.g. once with an aqueous solution comprising ethanol, and twice with a aqueous salt solution comprising ethanol, e.g. 80% ethanol and 50 mM sodium chloride), where each wash is loaded into the filter cartridge and passed through the filter by brief centrifugation. After the filtrate from the last wash is decanted, the filter cartridge is placed in the collection tube and centrifuged to remove the residual fluid. The filter cartridge is transferred to a fresh collection tube for elution of the RNA. The RNA is eluted by adding nuclease-free water (e.g. containing 0.1 mM EDTA), that is preheated above 80 degrees C. (preferably 95° C.), to the center of the silica filter.

### DETAILED DESCRIPTION OF PREFERRED EMBODIMENTS

In a preferred method of the present invention, paraffin-embedded tumor tissue should be sectioned, and the first slide section reviewed by a certified pathologist who is in close proximity to the paraffin-embedded block storage site (preferably the same building). Acceptable sections should be at least 75% tumor, as determined by the pathologist, unless exceptions are approved by the Principal Investigator. Each section should be 10 microns in width (plus or minus 10%), and a sample should consist of 24 sections in individual 2.0 mL cryovials labeled in the sequential order in which they were sliced. To the extent possible, samples should be treated as if they are fresh-frozen tissue (kept cold and dry, and preferably under inert gas), while avoiding freeze-thaw cycling. Samples should be shipped on dry ice, preferably the same day they are sliced, for next-day delivery. Shipment under liquid nitrogen is not desirable. Samples should be immediately blanketed under inert gas inside airtight zipped storage bags and stored at −80° C. until initiation of the sample extraction protocol.

All Sample Extraction procedures in a space/area designated specifically for RNA work only, within the pre-PCR laboratory. Equipment and consumables utilized in the Sample Extraction procedure should be reserved for use with this protocol alone, and clearly labeled “For RNA Work Only”. All consumables (i.e.: centrifuge tubes, pipette tips) must be sterile, RNase-free, and DNase-free, and designated as such by the vendor. RNaseZap® (commercially available from Applied Biosystems/Ambion) should be used liberally to thoroughly decontaminate and eliminate RNases from workspace and equipment surfaces prior to beginning every Sample Extraction procedure. Good laboratory practices should be used to prevent contamination with RNases, such as wearing a laboratory coat and changing gloves frequently.

It is preferred that a new DEPC-treated water bottle for every Sample Extraction procedure, to prevent contamination with RNases and cross-contamination with previous extractions. Upon receiving sectioned FFPE colorectal tumor tissue from the hospital site, in addition to following the Sample Handling procedure to properly store the samples, all sectioned tumor tissue should undergo Sample Extraction within 24 hours of being received (and no later than 48 hours of being received). Longer storage may compromise the fragile RNA template through oxidation of the sectioned tumor tissue, and is therefore not recommended.

As a pre-step to the protocol, one should preheat a benchtop heat block to 50° C. and another benchtop heat block to 70° C. From any single patient, remove no more than 8 cryovials containing individual FFPE tumor tissue sections of 10 microns width each from −80° C. storage. Preferably, one keeps the cryovials in dry ice on benchtop while working with patient sample. Next, one carefully taps tumor tissue sections from 8 cryovials into a single sterile 2.0 mL micro-centrifuge tube. Ideally, sectioned FFPE tumor tissue has formed tight curls; while cold and in a frozen state, sections should be easy to transfer to 2.0 mL micro-centrifuge tube. The total amount of tumor tissue sections per 2.0 mL micro-centrifuge tube should not exceed a total width of 80 microns (plus or minus 10%).

To remove the paraffin, add 1.00 mL of 100% xylene to each sample. Vortex briefly to mix. Incubate at 50° C. for 3 minutes to melt paraffin. After incubation, small amounts of paraffin may still be present. This is acceptable. If gross amounts of paraffin remain, consider repeating incubation for an additional 2 minutes.

Centrifuge for 2 minutes at maximum speed (12,000-15,000 rpm). Perform all centrifugation steps at room temperature. If sample does not form a tight pellet, repeat centrifugation for an additional 2 minutes. If pellet is still loose after second centrifugation, proceed with caution to next step.

Without disturbing pellet, carefully remove xylene with a pipette and discard into designated xylene/ethanol waste container. Sample will appear translucent and be difficult to see in this step. If pellet is loose, leave some xylene in micro-centrifuge tube and proceed. Most importantly, one should not remove or lose any tissue in an effort to remove xylene. Thereafter, add 1.00 mL of 100% ethanol (at room temperature) to each sample. Vortex briefly to mix. Centrifuge for 2 minutes at maximum speed (12,000-15,000 rpm). The sample will appear opaque/whitish after centrifugation with ethanol in this step. The sample should easily form a tight pellet. Without disturbing pellet, carefully remove ethanol with a pipette and discard into designated xylene/ethanol waste container. Repeat these last four steps for a second wash with another 1.00 mL of 100% ethanol. Briefly centrifuge sample again; carefully remove trace amounts of ethanol left with a pipette and discard. Air dry pellet at room temperature with micro-centrifuge tube tops open (it usually dries in approximately 15-20 minutes). Ensure ethanol is close to completely dried off before proceeding; otherwise, tissue digestion will be incomplete.

The sample should now be ready in order to proceed to the tissue digestion phase of the protocol. Another benchtop heat block is set to 70° C., while the other benchtop heat block is set to 50° C. Thereafter, add 400 μL of Digestion Buffer to each sample. Next, add 4.0 μL of Protease to each sample (protease is stored at −20° C.). Gently swirl or flick micro-centrifuge tube to fully immerse tissue. Ensure tissue is not stuck to side of micro-centrifuge tube above level of digestion solution. Do not vortex. If tissue will not become immersed, use a sterile pipette tip to dislodge it from wall of micro-centrifuge tube and to submerge into digestion solution. Incubate at 50° C. for 3 hours to isolate RNA. Sample mixture will appear fairly clear after 3 hours. If sample mixture still appears cloudy after incubation, tissue is probably heavily oxidized (damaged) and RNA yield and quality will be low.

Incubate at 70° C. for 20 minutes to break formaldehyde-induced cross-links between nucleic acids and proteins. See Li J, Smyth P, Cahill S, et al BMC Biotechnol. 2008 Feb. 6; 8:10. Set 50° C. benchtop heat block to 95° C., and incubate in it 2.0 mL of fresh DEPC-treated water in a micro-centrifuge tube in preparation for RNA elution (described below). If desired, the Sample Extraction procedure may be temporarily stopped this point, and samples stored at −20° C. When ready to continue, thaw samples on ice before proceeding.

Continuing on to RNA isolation, add 480 μL of Isolation Additive (from the Ambion Isolation Kit for FFPE) to each sample. Vortex to mix. The sample solution will appear white and cloudy in this step. Add 550 μL of 100% ethanol to each sample, and mix by carefully pipetting up and down. Add another 550 μL of 100% ethanol to each sample. Add carefully as total volume will be close to 2.0 mL after second ethanol addition. Mix by very carefully pipetting up and down. Sample solution will appear clear after ethanol addition in this step.

Place a Filter Cartridge (from the Ambion kit) into a Collection Tube for each sample to be processed. Add 700 μL of sample solution/mixture to Filter Cartridge. To prevent clogging of filter, avoid pipetting up large pieces of undigested tissue; smaller fragments are fine. Centrifuge for 2 minutes at 10,000×g (˜10,000 rpm). Do NOT centrifuge Filter Cartridge with sample at speeds greater than indicated; this will fracture Filter Cartridge.

Discard flow-through into waste container; reinsert Filter Cartridge into same Collection Tube. RNA becomes bound to Filter Cartridge after centrifugation. Repeat these three steps until all 2.0 mL of sample solution/mixture have been centrifuged through Filter Cartridge. This will take approximately 3 centrifugations.

Add 700 μL of Wash 1 to each Filter Cartridge. For unopened Wash 1 from new kits, add 42 mL of 100% ethanol (as indicated on bottle) to concentrate to bring up to working dilution. Centrifuge for 30 seconds at 10,000×g (˜10,000 rpm). Discard flow-through into waste container; reinsert Filter Cartridge into same Collection Tube. Add 500 μL of Wash 2/3 to each Filter Cartridge. For unopened Wash 2/3 from new kits, add 48 mL of 100% ethanol (as indicated on bottle) to concentrate to bring up to working dilution. Centrifuge for 30 seconds at 10,000×g (˜10,000 rpm). Discard flow-through into waste container; reinsert Filter Cartridge into same Collection Tube. Centrifuge for another minute to remove residual amounts of Wash solutions from Filter Cartridge.

Make a master mix of DNA Digestion reagents, sufficient for all samples being processed plus 1-2 extra (pipetting excess), in the following ratio: for one sample, use 50 ul DEPC-Treated Water, 6 ul (10×) DNAse buffer, and 4 ul DNAse (thus, for two samples, these amounts are doubled, etc.). Add 60 μL of DNase master mix to center of each Filter Cartridge. Close Collection Tube tops; incubate at room temperature for 30 minutes to digest DNA.

In order to purify the RNA, add 700 μL of Wash 1 to each Filter Cartridge. Let the sample sit at room temperature for 1 minute. Centrifuge for 30 seconds at 10,000×g (10,000 rpm). Discard flow-through into waste container; reinsert Filter Cartridge into same Collection Tube. Add 500 μL of Wash 2/3 to each Filter Cartridge. Centrifuge for 30 seconds at 10,000×g (10,000 rpm). Discard flow-through into waste container; reinsert Filter Cartridge into same Collection Tube. Repeat these last three steps for a second wash with another 500 μL of Wash 2/3. Centrifuge for another minute at 10,000×g (10,000 rpm) to remove residual amounts of Wash solutions from Filter Cartridge.

The protocol can now proceed to RNA elution. For this purpose, transfer Filter Cartridge to fresh Collection Tube. Apply 50 μL of DEPC-treated water heated to 95° C. to center of each Filter Cartridge. Use a P200 pipette and appropriate sterile pipette tip, and insert only the edge of the tip into the heated DEPC-treated water. The high temperature will cause air inside the pipette tip to expand, and therefore unexpectedly expel aspirated water if the pipette tip is heated too much. This is avoidable by inserting as little of the pipette tip's surface into the water as possible, and moving quickly to the Filter Cartridge. Close the Collection Tube tops and incubate at room temperature for 1 minute to hydrate bound RNA. Centrifuge for 1 minute at 10,000×g (10,000 rpm). Repeat these three steps for a second elution with another 50 μL of DEPC-treated water heated to 95° C. The final volume of eluted RNA will be approximately 85 μL (the reduction due to vapor loss). The second elution may not be necessary when processing only 4 FFPE tumor tissue sections of 10 microns width each (total width of 40 microns). If desired, when working with this smaller amount of material, one can elute RNA with only 1 aliquot of 50 μL of DEPC-treated water heated to 95° C. After elution, discard Filter Cartridge and close Collection Tube tops. Store RNA samples at −80° C., or place on ice for immediate quantitation.

### EXPERIMENTAL

The following examples are only intended as illustrative and are not intended to provide any limitations to the present invention.

### Example 1

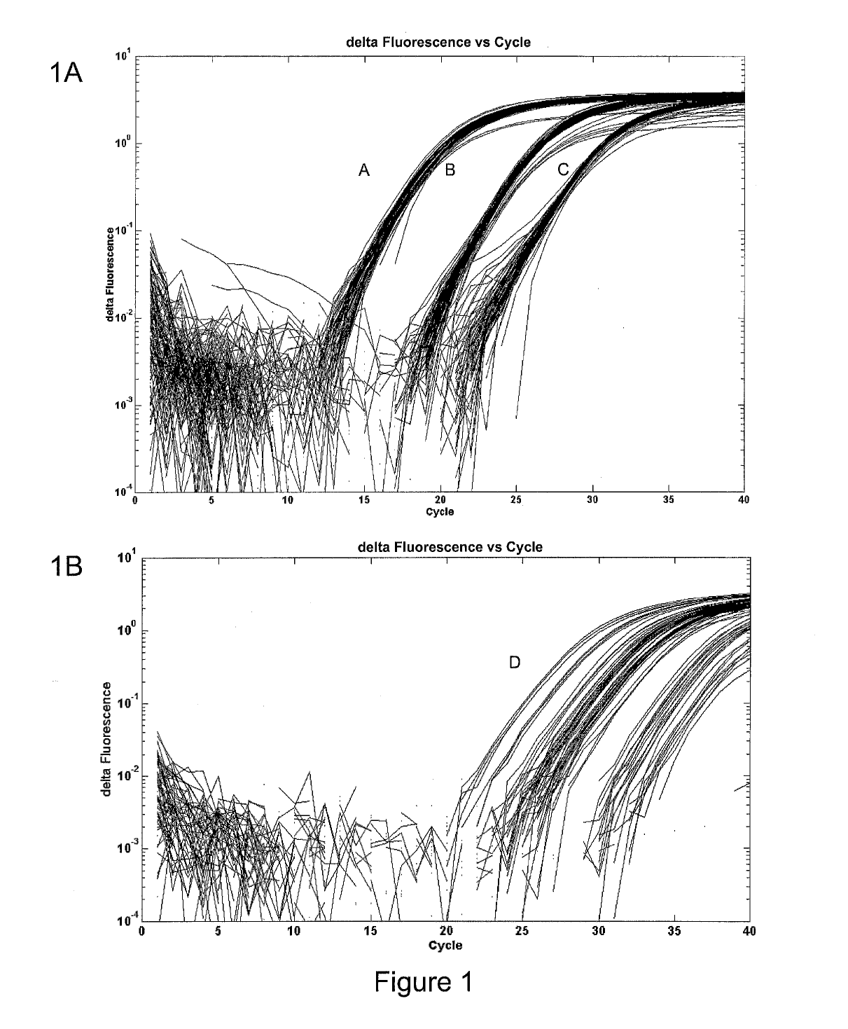

In the course of performing our initial validation study, we noted the substantial variability between plates. We investigated the possibility of fixing the sample concentration and normalizing to an endogenous control to correct this. We compared this method with the use of our internally designed non-human controls (FIG. 1). As shown, the endogenous control (FIG. 1B) varies far more widely than expected for inter-run variability. This is likely a result of individual expression levels and high variability in overall sample quality, which is affected by many factors. Our spike in controls performed better (FIG. 1A), showing only the expected variability between runs. Being able to control this variability is important in detecting the subtle expression differences present in the recurrent and non-recurrent disease states. Further, use of a sample-independent control is the most effective method for identifying inter-test variability.

1. A method of improving human RNA yield, comprising:
a) providing human RNA released from formalin-fixed paraffin-embedded colorectal cancer tissue;
b) recovering said RNA by solid phase extraction on a filter comprising i) loading the RNA on said filter, ii) washing the filter, and iii) eluting the RNA, wherein said eluting comprises applying an aqueous solution to said filter, wherein said solution has been heated above 80° C.

2. The method of claim 1, wherein said solution has been heated to approximately 95° C.

3. The method of claim 1, wherein said filter is part of a filter cartridge.

4. The method of claim 1, wherein said filter comprises silica.

5. The method of claim 1, wherein the yield of RNA eluted from said filter with said heated solution is higher than achieved with an unheated solution.

6. The method of claim 2, wherein the volume of solution added to the filter is between 50 and 100 microliters.

7. The method of claim 1, further comprising utilizing said RNA in an RT-PCR assay.

8. The method of claim 7, wherein said RT-PCR assay generates amplicons between 60 and 150 bases in length.

9. The method of claim 7, wherein said RT-PCR assay generates amplicons from non-human control sequences.

10. The method of claim 9, wherein said non-human control sequences are selected from the group consisting of SEQ ID NOS: 1-3.

11. A method of controlling for variability in an RT-PCR assay, comprising:
a) providing human RNA released from formalin-fixed paraffin-embedded colorectal cancer tissue; and
b) utilizing said RNA in an RT-PCR assay comprising spiked-in controls.

12. The method of claim 11, wherein said RT-PCR assay generates amplicons from said spiked in controls.

13. The method of claim 12, wherein said spiked-in controls are non-human control sequences.

14. The method of claim 13, wherein said non-human control sequences are selected from the group consisting of SEQ ID NOS: 1-3.


SEQUENCE LISTING (3 sequences)

SEQ ID NO: 1 - DNA, 100 bp, Drosophila melanogaster
ccttccccgatcacaatcagagtccgcgtaacacctatcaaagcgctatgggtaagcaagctatgggcgtttatattaccaacttccacgtgcgtatgga

SEQ ID NO: 2 - DNA, 100 bp, Drosophila melanogaster
agcgcaccaagcacttcatccgccaccagtcggatcgatatgctaagctgtcgcacaaatggcgcaagcccaagggtatcgacaacagagtgcgtcgacg

SEQ ID NO: 3 - DNA, 110 bp, Arabidopsis
acctgcagcagcacccgcaagtggtcctaatgcaaatccgttagatctcttcccacagggcttgccaaatgttggaggaaatcctggtgctggaacacttgacttcttgc